(19) United States
(12) Patent Application Publication
(10) Pub. No.: US 2026/0195770 A1
(43) Pub. Date: Jul. 9, 2026

(54) EMISSIONS MONITORING AND EMISSIONS SOURCE IDENTIFICATION

(71) Applicant: GM GLOBAL TECHNOLOGY OPERATIONS LLC, DETROIT, MI (US)

(72) Inventor: Mohammad Manjurul Islam, Ajax (CA)

(21) Appl. No.: 19/014,782

(22) Filed: Jan. 9, 2025

(51) Int. Cl. G06Q 30/018; F01N 11/00; G07C 5/08

(52) U.S. Cl. CPC G06Q 30/018; F01N 11/007; G07C 5/0808; G07C 5/0816

(57) ABSTRACT

A method of monitoring emissions from a vehicle includes receiving real time measurement data from an emissions sensor, the measurement data including a detected amount of a constituent of the emissions from the vehicle. The method also includes inputting the measurement data to a machine learning model, the machine learning model configured to correlate the measurement data and at least one operating parameter with a source of the detected amount of the constituent, and predicting the source of the detected amount of the constituent based on the machine learning model.

### INTRODUCTION

The subject disclosure relates to combustion engines, and more particularly to monitoring emissions from a vehicle.

Internal combustion engine (ICE) vehicles, including traditional combustion vehicles and hybrid vehicles, feature systems for monitoring emissions. The ability to accurately assess emissions is important in order to reduce pollutants and limit the environmental impact of ICE vehicles. It is desirable to provide systems and methods that can accurately measure constituents of vehicle exhaust and determine sources of such constituents, in order to further reduce the environmental impact of ICE vehicles and combustion engines more generally.

### SUMMARY

In one exemplary embodiment, a method of monitoring emissions from a vehicle includes receiving real time measurement data from an emissions sensor, the measurement data including a detected amount of a constituent of the emissions from the vehicle. The method also includes inputting the measurement data to a machine learning model, the machine learning model configured to correlate the measurement data and at least one operating parameter with a source of the detected amount of the constituent, and predicting the source of the detected amount of the constituent based on the machine learning model.

In addition to one or more of the features described herein, the method includes, based on predicting the source, performing at least one of presenting information related to the measurement data and the predicted source to a user, presenting a suggestion to the user for adjusting operation of the vehicle, providing a route suggestion, and autonomously controlling the vehicle.

In addition to one or more of the features described herein, the detected amount of the constituent includes at least one of a concentration of the constituent and a chemical profile.

In addition to one or more of the features described herein, the source of the detected amount of the constituent is an operating condition of a component of the vehicle.

In addition to one or more of the features described herein, the method includes receiving real time environmental monitoring data from an external monitoring device.

In addition to one or more of the features described herein, the method includes determining a route of the vehicle based on the detected amount of the constituent and the environmental monitoring data.

In addition to one or more of the features described herein, the predicted source is a component of the vehicle, and the method further comprises communicating with an inspection device associated with the component, and causing the inspection device to perform a diagnostic on the component.

In addition to one or more of the features described herein, the at least one operating parameter includes a type of the vehicle, and the method further comprises associating the predicted source with the type of the vehicle.

In addition to one or more of the features described herein, the method includes storing at least one of the measurement data and information related to the predicted source in a blockchain-based system.

In another exemplary embodiment, a system for monitoring emissions from a vehicle includes a machine learning module configured to receive real time measurement data from an emissions sensor, the measurement data including a detected amount of a constituent of emissions from the vehicle, the machine learning module configured to input the measurement data to a machine learning model. The machine learning model is configured to correlate the measurement data and at least one operating parameter with a source of the detected amount of the constituent, and predict the source of the detected amount of the constituent. The system also includes an inspection device associated with a component of the vehicle, the inspection device configured to, based on the source of the detected amount of the constituent being an operating condition of the component, perform a diagnostic on the component. The system further includes a device configured to communicate the predicted source and a result of the diagnostic to at least one of a vehicle user and an external entity.

In addition to one or more of the features described herein, the device includes a user interface configured to perform at least one of: presenting information related to the predicted source, presenting a suggestion for adjusting operation of the vehicle, and providing a route suggestion.

In addition to one or more of the features described herein, the detected amount of the constituent includes at least one of: a concentration of the constituent and a chemical profile.

In addition to one or more of the features described herein, the device is configured to receive real time environmental monitoring data from an external monitoring device.

In addition to one or more of the features described herein, the device is configured to determine a route of the vehicle based the detected amount of the constituent and the environmental monitoring data.

In addition to one or more of the features described herein, the at least one operating parameter includes a type of the vehicle, and the system is configured to associate the predicted source with the type of the vehicle.

In addition to one or more of the features described herein, the system is configured to store at least one of the measurement data and information related to the predicted source in a blockchain-based system.

In yet another exemplary embodiment, a vehicle system includes an emissions sensor coupled to an exhaust system of a vehicle, and a machine learning module configured to receive real time measurement data from the emissions sensor, the measurement data including a detected amount of a constituent of emissions from the vehicle, the machine learning module configured to input the measurement data to a machine learning model. The machine learning model is configured to correlate the measurement data and at least one operating parameter with a source of the detected amount of the constituent, and predict the source of the detected amount of the constituent. The vehicle system also includes an inspection device associated with a component of the vehicle, the inspection device configured to, based on the source of the constituent being an operating condition of the component, perform a diagnostic on the component.

In addition to one or more of the features described herein, the vehicle system includes a user interface configured to perform at least one of: presenting information related to the predicted source, presenting a suggestion for adjusting operation of the vehicle, and providing a route suggestion.

In addition to one or more of the features described herein, the vehicle system includes a device configured to receive real time environmental monitoring data from an external monitoring device.

In addition to one or more of the features described herein, the device is configured to determine a route of the vehicle based the detected amount of the constituent and the environmental monitoring data.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings in which:

FIG. 1 is a top view of a motor vehicle including an emissions monitoring system, in accordance with an exemplary embodiment;

FIG. 2 schematically depicts a vehicle system including components for emission monitoring and emission source identification, in accordance with an exemplary embodiment;

FIG. 3 depicts a machine learning module for identifying emission sources, in accordance with an exemplary embodiment;

FIG. 4 is a flow diagram representing aspects of a method of monitoring and evaluating combustion engine emissions, in accordance with an exemplary embodiment; and

FIG. 5 depicts a computer system, in accordance with an exemplary embodiment.

### DETAILED DESCRIPTION

The following description is merely exemplary in nature and is not intended to limit the present disclosure, its application, or uses. It should be understood that throughout the drawings, corresponding reference numerals indicate like or corresponding parts and features.

Devices, systems and methods are provided for monitoring vehicle and/or combustion engine emissions. An embodiment of a system includes a processor configured to receive measurement data from one or more emissions sensors. Examples of such sensors include chemical sensors capable of detecting concentrations of chemical constituents of exhaust gases.

The system includes a machine learning module that includes a machine learning model, such as a neural network, which is trained on chemical spectral profiles (and/or other data describing constituents of measured vehicle emissions) and vehicle operational parameters. The machine learning model is configured to provide real time predictions that identify sources of measured constituents (referred to as “emission sources”).

The system may include additional components, such as vehicle-integrated inspection devices configured to inspect and perform diagnostics on identified emission sources, and a user interface for providing real time information and feedback to a user. In an embodiment, the system is configured to securely store data using blockchain technology. The system may communicate with external devices and systems, such as environmental monitoring devices.

Embodiments described herein present numerous advantages and technical effects. The invention provides a comprehensive and scalable solution for real-time monitoring and identification of vehicle emissions. For example, embodiments can provide continuous, real-time monitoring of vehicle emissions, allowing for immediate detection and response to emission issues. This is a significant improvement over periodic inspections, which can miss transient emission events. Embodiments provide for vehicle-integrated inspection devices that allow for on-the-spot inspections and dynamic adjustments to vehicle operation, allowing for prompt addressing of emission problems and corresponding reductions in environmental impact.

Embodiments also allow for integrating emissions monitoring and source identification, by connecting with other environmental monitoring devices (e.g., pollution monitoring stations at various geographic locations). This integration allows for achieving a holistic view of environmental conditions. This enhances the system's ability to identify and address emission sources effectively, and also provides opportunities to avoid regions or populations that may be sensitive to certain emissions constituents.

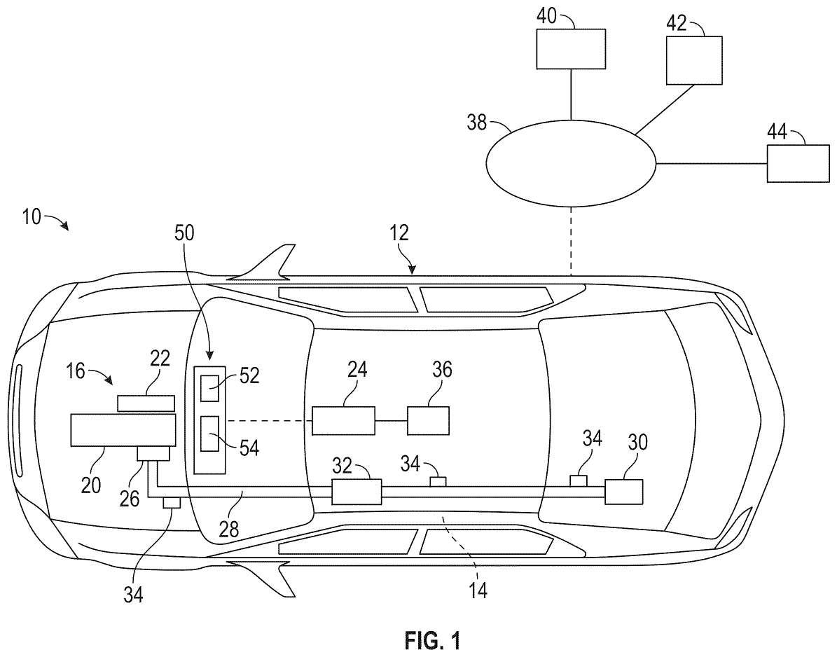

FIG. 1 shows an embodiment of a motor vehicle 10, which includes a vehicle body 12 defining, at least in part, an occupant compartment 14. The vehicle body 12 also supports various vehicle subsystems including a propulsion system 16, and other subsystems to support functions of the propulsion system 16 and other vehicle components, such as a fuel system, a braking system, a suspension system, a steering subsystem and others.

The vehicle may be a combustion engine vehicle or a hybrid vehicle. In an example, the vehicle 10 is a hybrid vehicle that includes a combustion engine 20 and an electric motor 22.

The vehicle also includes various control systems for controlling aspects of vehicle systems. For example, one or more electronic control units (ECUs) 24 are provided. Aspects of the various methods described herein may be performed by any suitable controller or processing device, such as the ECU 24 and/or one or more other controllers in respective subsystems.

The vehicle 10 also includes an exhaust system, which includes an exhaust manifold 26, an exhaust pipe (or pipes) 28 and an outlet 30. The exhaust system may include other components, such as a catalytic converter 32 configured to reduce pollutants, a muffler and others.

The exhaust system also includes one or more sensors 34 (referred to as emissions sensors) configured to detect one or more constituents of exhaust produced by the engine 20. FIG. 1 shows an example in which multiple emissions sensors 34 are disposed at multiple locations for monitoring emissions. It is noted that embodiments are not so limited, as there may be any number of emissions sensors (i.e., one or more) at any number of locations.

An emissions sensor, in an embodiment, is configured to detect one or more constituents of a vehicle's exhaust. For example, the emissions sensor may be a nitrous oxide sensor, a carbon dioxide sensor and/or other suitable type of sensor. Examples of exhaust constituents include oxygen, nitrous oxide, carbon monoxide, carbon dioxide, water, particulate matter and others. Each emissions sensor may be configured to measure a concentration or chemical profile of a constituent.

In an embodiment, at least one emissions sensor is an advanced chemical sensor capable of high sensitivity and accuracy. Such sensors are capable of providing highly accurate chemical readings in a short time, allowing for effective real time monitoring. Integration of such sensors can achieve high accuracy in detecting and identifying emission sources. This reduces false positives and ensures that only genuine emission issues are flagged.

Any combination of one or more sensors, such as chemical sensors configured to identify specific chemicals in vehicle exhaust, may be used. Examples include non-dispersive Infrared (NDIR) sensors, electrochemical sensors, metal oxide semiconductor (MOS) sensors, photoionization detectors (PID), pellistor (catalytic bead) sensors, and others. One or more sensors (e.g., as individual sensor devices or modules, or as a combined sensor) may be incorporated into the vehicle 10.

NDIR sensors are used to measure constituent gases such as carbon monoxide (CO), carbon dioxide (CO2), and hydrocarbons (HC). NDIR sensors work by detecting the absorption of infrared light at specific wavelengths corresponding to different gases.

Electrochemical sensors are used to detect gases such as oxygen (O2) and nitrogen oxides (NOx), and operate by generating a current proportional to the concentration of the target gas. MOS sensors are used for detecting volatile organic compounds (VOCs) and other gases. MOS sensors change their electrical resistance in the presence of specific gases.

PID sensors are effective for detecting VOCs. PID sensors use ultraviolet light to ionize gas molecules, which are then detected by measuring the resulting current. Pellistor sensors are used to detect combustible gases, and operate by oxidizing gas on a catalytic bead to cause a change in temperature that is measured to determine gas concentration.

An embodiment of the vehicle 10 includes devices and/or systems for communicating with other vehicles and/or objects external to the vehicle. For example, the vehicle 10 includes a communication system having a telematics unit 36 or other suitable device including an antenna or other transmitter/receiver for communicating with a network 38.

The network 38 represents any one or a combination of different types of suitable communications networks, such as public networks (e.g., the Internet), private networks, wireless networks, cellular networks, or any other suitable private and/or public networks. Further, the network 38 can have any suitable communication range associated therewith and may include, for example, global networks (e.g., the Internet), metropolitan area networks (MANs), wide area networks (WANs), local area networks (LANs), or personal area networks (PANs). The network 38 can communicate via any suitable communication modality, such as short range wireless, radio frequency, satellite communication, or any combination thereof.

In an embodiment, the network 38 connects with the vehicle 10 for communication with various entities. For example, the network 38 may be connected to a server 40, a database 42 and/or one or more other remote entities 44 such as workstations, control centers, environmental monitoring systems, other vehicles and others.

The vehicle 10 also includes a computer system 50 that includes one or more processing devices 52 and a user interface 54. The various processing devices and units may communicate with one another via a communication device or system, such as a controller area network (CAN) or transmission control protocol (TCP) bus.

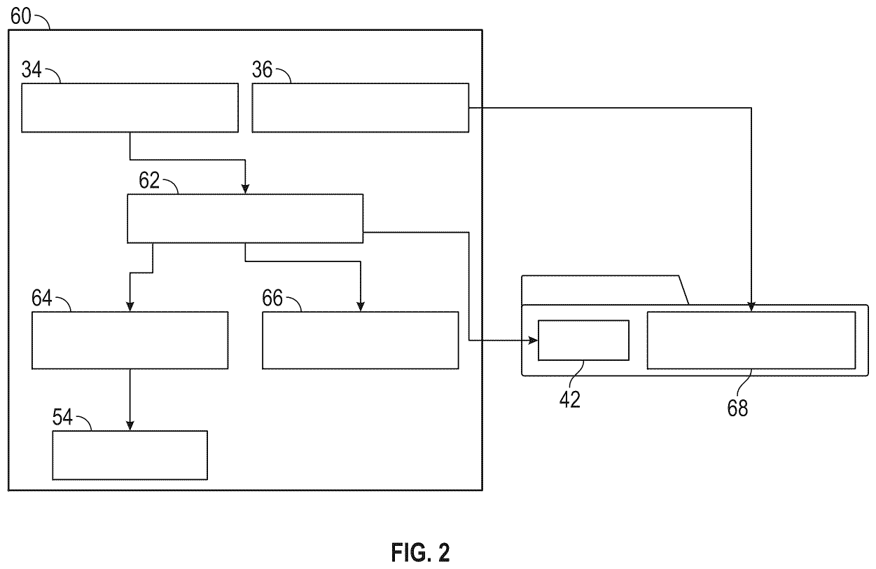

FIG. 2 schematically depicts an embodiment of a vehicle system 60, which includes various components for performing functions related to emissions monitoring. Generally, emissions monitoring as described herein includes receiving measurement data from one or more sensors 34. The measurement data indicates, in an embodiment, a concentration of a chemical constituent or constituents (e.g., nitrous oxide, oxygen, carbon dioxide, etc.). For example, one or more of the sensors is a chemical sensor that provides a spectral chemical profile. The measurement data may be for a given time or over a selected time window.

Based on the measurement data, a machine learning model or algorithm is used to identify an emission source considered to be responsible for, or contributing to, emission of a detected constituent. An “emission source” refers to any device, system or condition that is considered to be a cause of the detected constituent. Examples include faulty engine components, a faulty, worn or damaged catalytic converter, or other vehicle component.

The vehicle system 60 includes various modules for performing aspects of methods described herein. In an embodiment, the vehicle system 60 includes a machine learning module 62 that executes a machine learning model or algorithm. The machine learning module 62 receives measurement data from the sensor(s) 34, and other information such as vehicle type (e.g. passenger, truck, specific vehicle model, etc.), operational parameters (e.g., engine speed, torque, etc.) and any other relevant information, such as weather conditions. Based on this information, the machine learning module 62 performs a classification process to attempt to identify an emissions source.

Upon identification of an emission source, the machine learning model outputs to one or more other devices or processors, for performing various actions. For example, the emissions source may be output to the ECU 24 (FIG. 1). The ECU 24 performs actions such as controlling parameters of the engine 20, such as reducing engine speed.

In an example, an action includes controlling an engine operating parameter range (e.g., rotational speed and/or torque range), so as to avoid operating parameter values associated a highest level of emission of a given constituent or constituents.

In another example, outputs from the machine learning module 62 are provided to an optimization process that optimizes or controls operating parameters using a multi-criteria optimization to reduce emissions and improve performance. For example, an optimization process is performed that considers criteria such as emission level, cost and operational efficiency. An example of an optimization process determines operating parameters that reduce or minimize emissions (e.g., to reduce the total emissions from a vehicle fleet), increase cost efficiency (e.g., reduce or minimize costs associated with emission reduction strategies, including maintenance and fuel costs), and maintain or increase operational efficiency (e.g., ensure that the optimization does not negatively impact the operational efficiency of the vehicle fleet).

In an embodiment, the vehicle system 60 includes one or more onboard inspection devices 64. Each inspection device 64 is connected to a propulsion system or other vehicle system component, such as an engine component, the catalytic converter 32 (FIG. 1), an exhaust system component and others. If a component is identified as an emissions source, the machine learning module 62 may output emission source information to an appropriate inspection device 64, a request to the inspection device 64, or otherwise prompt the inspection device 64 to perform a diagnostic to determine whether the component is faulty or is otherwise operating in a sub-optimal manner.

The vehicle system 60 may perform or facilitate various actions, such as presenting emissions information and emission source predictions, providing suggestions regarding changes in driving style and/or planned routes, and automated control of the vehicle 10 (e.g., fully autonomous or driver assist).

In an embodiment, the system 60 is configured to store information related to monitoring and source identification to a blockchain system. For example, a blockchain-based data logging module 66 is configured to format data (e.g., log data from the sensor(s) 34) and transmit the formatted data to a blockchain system. Using blockchain technology for data logging ensures that emission data is secure, transparent, and tamper-proof. This is useful, for example, for regulatory compliance and environmental reporting.

The vehicle 10, in an embodiment, is configured to communicate with various external devices, such as a database and various environmental monitoring systems. For example, the vehicle 10 can communicate with the database 42 for storage and retrieval of measurement data, such as chemical spectral profiles. The database may also store other information, such as vehicle type and environmental conditions.

The environmental monitoring system includes one or more environmental monitoring sensors 68 at various geographic locations for detecting pollutants, monitoring weather and climate conditions, and others. Information from the sensors 68 (e.g., temperature, weather, time of day, season etc.) can be input to the machine learning model for source prediction.

Information from the sensors (e.g., pollution type and levels) and/or other information (e.g., locations of sensitive sites such as hospitals) can also be used for route planning. For example, if a spectral profile or other measurement data is indicative of high levels of a certain pollutant, the vehicle system 60 may provide route suggestions to avoid sensitive sites via the interface 54.

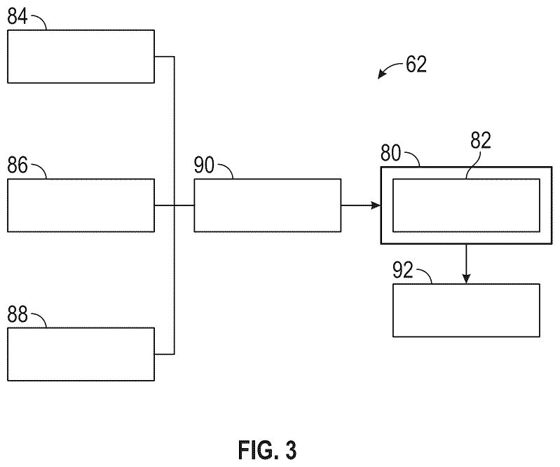

FIG. 3 depicts an embodiment of the machine learning module 62. The machine learning module 62 utilizes a neural network or other machine learning model, which is trained to determine probabilities of various emissions sources based on a chemical spectrum (or other data related to a concentration or amount of a detected constituent) received from a sensor or sensors 34. Although the measurement data is described as being chemical spectra, the measurement data is not so limited.

The machine learning module 62 includes a machine learning model 80. The machine learning model 80, in an embodiment, includes at least one feature space or embedding space 82.

The machine learning model 80 receives input data that includes chemical profiles 84 (and/or other measurement data), and operational data 86 representing operational parameters such as speed, acceleration and braking. Other information 88, such as weather and other environmental information, vehicle type and others, may also be input to the model 80.

The machine learning module 62 is configured to convert received data into feature vectors 90, which are input to the model 80. The model outputs emission source data 92, such as an identification of a given emission source, and/or a probability that a given emission source is a cause of a detected constituent (or is a contributing factor). In an embodiment, the machine learning module 62 converts the measurement data and operational parameters to a feature vector 90 that is input to the model 80 for emission source identification and/or training.

The following is an example of the feature vector 90, denoted as X. The feature vector includes a concentration feature Cᵢ, where C represents a concentration or amount of a given constituent, and i represents a sensor index (i.e., an identifier of a given sensor 34). The feature vector 90 also includes an operational parameter feature Vj, where V represents an operational parameter (e.g., speed, engine load, etc.) and j identifies a specific parameter. The feature vector 90 may be represented by:

$$X = [ {C}_{1} , {C}_{2} , … {C}_{m} , {V}_{1} , {V}_{2} , … , {V}_{n} ] ,$$

where m represents a number of concentration features (e.g., a number of different chemicals or constituents) and n represents a number of different operational parameters.

The machine learning model 80 maps the feature vector X to a probability P=f(X) of each of one or more potential emission sources. An “identified” emissions source may be a source having the highest probability.

The machine learning module 62 may continuously or periodically update sensor readings and vehicle operation data, and train the model 80 to further improve and refine predictions. For example, the feature vector 90 may be represented by:

$$X ( t ) = [ {C}_{1} ( t ) , {C}_{2} ( t ) , … {C}_{m} ( t ) , {V}_{1} ( t ) , {V}_{2} ( t ) , … , {V}_{n} ( t ) ] ,$$

where t is time.

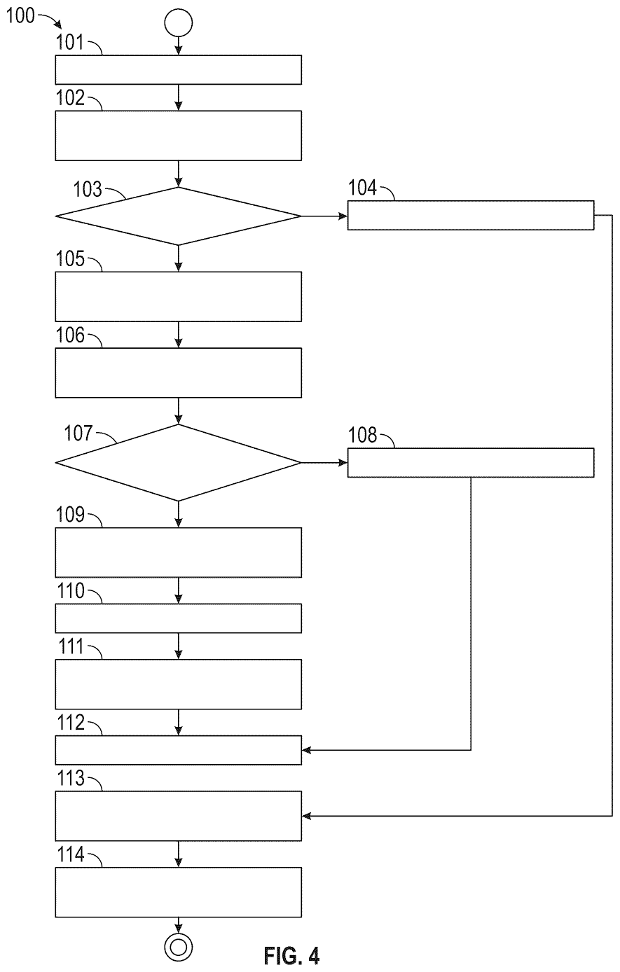

FIG. 4 illustrates embodiments of a method 100 of controlling a combustion engine. Aspects of the method 100 may be performed by the ECU 24, the machine learning module 62 and/or other suitable processing device or combination of processing devices.

The method 100 is described in conjunction with the vehicle 10 of FIG. 1 and the vehicle system 60 of FIG. 2 for illustrative purposes. Embodiments are not so limited, as the method 100 may be performed in conjunction with any suitable system that utilizes a combustion engine.

The method 100 includes a number of steps or stages represented by blocks 101-114. The method 100 is not limited to the number or order of steps therein, as some steps represented by blocks 101-114 may be performed in a different order than that described below, or fewer than all of the steps may be performed.

At block 101, the method 100 begins with monitoring vehicle operation and collecting data related to operational parameters, such as speed and engine load. Other information may be collected as discussed above.

At block 102, measurement data, such as one or more chemical profiles, is received from each sensor 34. The measurement data may be collected continuously or periodically during operation of the vehicle 10.

At block 103, it is determined whether sufficient data has been collected. If not, data collection is retried at block 104. If the retried data collection is successful, an external entity may be accessed (block 113), for example, to externally store or transmit collected data.

At block 105, if sufficient data has been collected, the operational data and measurement data are input to the machine learning module 62. The machine learning module 62 analyzes the operational and measurement data as described herein as part of a classification or other machine learning operation to identify an emission source (block 106).

At block 107, the machine learning module 62 determines whether an emission source has been identified. If not, monitoring continues at block 108.

At block 109, if an emission source is identified as a vehicle component (e.g., an engine component, the catalytic converter, etc.), emission source information and/or a request is sent to an appropriate inspection device 64.

An example of a detected constituent or pollutant associated with an emission source is nitrogen oxide (NOX). NOx pollutants are typically associated with high-temperature combustion in engines. For instance, when a vehicle is accelerating or climbing a hill, the engine operates at higher temperatures, leading to increased NOx emissions. To mitigate this, vehicles often use technologies like Exhaust Gas Recirculation (EGR), which reduces the formation of NOx by recirculating a portion of exhaust gases back into engine cylinders, lowering the combustion temperature.

Another example is particulate matter (PM), which is produced during incomplete combustion of fuel, especially in diesel engines. High PM emissions are often observed during conditions such as cold starts, idling, and heavy acceleration. To reduce PM emissions, vehicles use Diesel Particulate Filters (DPF), which trap and oxidize particulate matter in the exhaust system.

At block 110, the inspection device 64 inspects the identified component. At block 111, a diagnostic report or other indication as to whether the component is operating normally is sent to the user interface 54. The user interface 54 may then present emission source information to a user (e.g., a driver, a remote technician, etc.).

It is noted that multiple emission sources may be identified. For example, if more than one emissions source has a high probability (e.g., a probability that meets or exceeds a threshold, such as 50%, 75%, etc.), the machine learning module 62 may output emission source information and/or requests to an inspection device for each identified emission source.

The user interface 54 may present suggestions to the driver as to actions that the driver can perform in response to detection of an emission source. For example, the user interface can present suggestions to the driver to change driving style to reduce the emission of a constituent, change the fuel grade (octane) or add suggested fuel additives. In another example, the user interface 54 presents a planned route based on identified emissions sources. The planned route is determined such that sensitive locations, such as hospital, schools and nursing homes, are avoided.

At block 112, results of emission monitoring are stored in a desired location, such as the database 42, for further training. In an embodiment, the results are stored as part of a blockchain. The blockchain allows for the source of data to be identified, and prevents modification so as to preserve a historical record.

At block 113, the vehicle system 60 (e.g., via the telematics unit 36) may access external entities, such as the database 42, other vehicles, remote monitoring centers, fleet management, dealerships, technicians and others.

At block 114, chemical profiles and other emissions source information is accessed (e.g., from the database 42) by any desired entity. Manufacturers, dealers, environmental monitoring systems and/or other entities can access the information to allow for determination of emissions profiles for various vehicle types, to allow for fleet management and others. For example, manufacturers can use emissions profiles associated with specific models to improve designs.

As noted herein, any of one or more actions may be performed based on emission source information. For example, operating parameters such as engine rotational speed, vehicle speed, acceleration limits and others are controlled to mitigate emission of undesired pollutants and/or other constituents. Operating parameters may be controlled via an optimization algorithm as described herein. Other actions include adding, activating and/or controlling one or more emission control systems. Examples of emission control systems include catalytic converters, air injection systems, diesel oxidation catalyst systems (DOC), EGRs, DPFs and others.

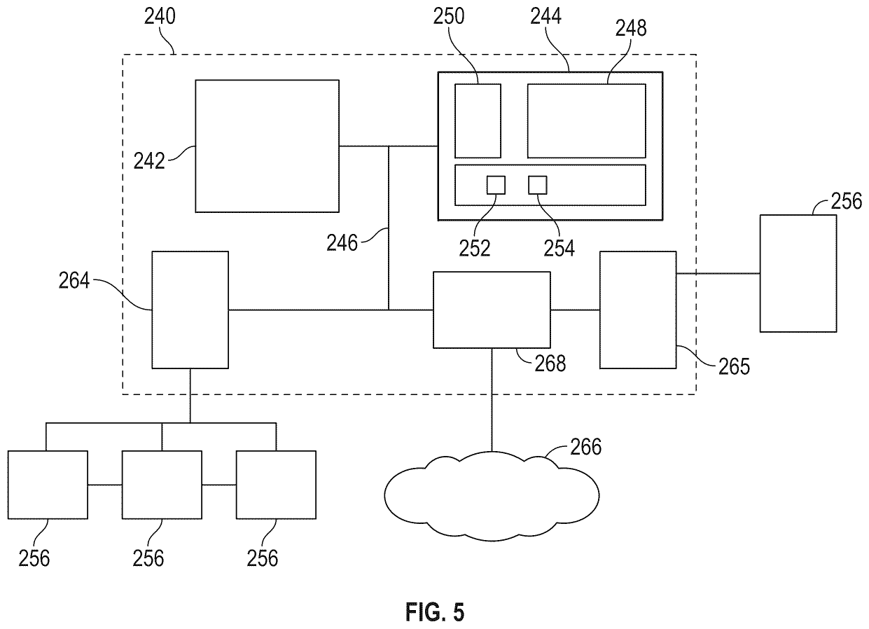

FIG. 5 illustrates aspects of an embodiment of a computer system 240 that can perform various aspects of embodiments described herein. The computer system 240 includes at least one processing device 242, which generally includes one or more processors for performing aspects of methods described herein.

Components of the computer system 240 include the processing device 242 (such as one or more processors or processing units), a memory 244, and a bus 246 that couples various system components including the system memory 244 to the processing device 242. The system memory 244 can be a non-transitory computer-readable medium, and may include a variety of computer system readable media. Such media can be any available media that is accessible by the processing device 242, and includes both volatile and non-volatile media, and removable and non-removable media.

For example, the system memory 244 includes a non-volatile memory 248 such as a hard drive, and may also include a volatile memory 250, such as random access memory (RAM) and/or cache memory. The computer system 240 can further include other removable/non-removable, volatile/non-volatile computer system storage media.

The system memory 244 can include at least one program product having a set (i.e., at least one) of program modules that are configured to carry out functions of the embodiments described herein. For example, the system memory 244 stores various program modules that generally carry out the functions and/or methodologies of embodiments described herein. A module 250 may be included for performing functions related to acquiring signals and data, and a module 252 may be included to perform functions related to emissions source identification and diagnostics as discussed herein. The system 240 is not so limited, as other modules may be included. As used herein, the term “module” refers to processing circuitry that may include an application specific integrated circuit (ASIC), an electronic circuit, a processor (shared, dedicated, or group) and memory that executes one or more software or firmware programs, a combinational logic circuit, and/or other suitable components that provide the described functionality.

The processing device 242 can also communicate with one or more external devices 256 as a keyboard, a pointing device, and/or any devices (e.g., network card, modem, etc.) that enable the processing device 242 to communicate with one or more other computing devices. Communication with various devices can occur via Input/Output (I/O) interfaces 264 and 265.

The processing device 242 may also communicate with one or more networks 266 such as a local area network (LAN), a general wide area network (WAN), a bus network and/or a public network (e.g., the Internet) via a network adapter 268. It should be understood that although not shown, other hardware and/or software components may be used in conjunction with the computer system 40. Examples include, but are not limited to: microcode, device drivers, redundant processing units, external disk drive arrays, RAID systems, and data archival storage systems, etc.

The terms “a” and “an” do not denote a limitation of quantity, but rather denote the presence of at least one of the referenced item. The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

When an element such as a layer, film, region, or substrate is referred to as being “on” another element, it can be directly on the other element or intervening elements may also be present. In contrast, when an element is referred to as being “directly on” another element, there are no intervening elements present.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this invention belongs.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

1. A method of monitoring emissions from a vehicle, comprising:
receiving real time measurement data from an emissions sensor, the measurement data including a detected amount of a constituent of the emissions from the vehicle;
receiving real time environmental monitoring data from an external monitoring device;
inputting the measurement data and the environmental monitoring data to a machine learning model, the machine learning model configured to correlate the measurement data and at least one operating parameter with a source of the detected amount of the constituent;
predicting the source of the detected amount of the constituent based on the machine learning model; and
based on predicting the source, autonomously controlling the vehicle to reduce the emissions.

2. The method of claim 1, further comprising, based on predicting the source, performing at least one of presenting information related to the measurement data and the predicted source to a user, presenting a suggestion to the user for adjusting operation of the vehicle, and providing a route suggestion.

3. The method of claim 1, wherein the detected amount of the constituent includes at least one of a concentration of the constituent and a chemical profile.

4. The method of claim 1, wherein the source of the detected amount of the constituent is an operating condition of a component of the vehicle.

5. The method of claim 1, wherein the constituent of the emissions includes a pollutant, and the method further comprises:
comparing the detected amount of the constituent to a threshold amount; and
based on the detected amount exceeding the threshold amount, performing at least one of: autonomously controlling the vehicle to avoid a sensitive site, and suggesting a route to the user that avoids the sensitive site.

6. The method of claim 1, further comprising determining a route of the vehicle based on the detected amount of the constituent and the environmental monitoring data.

7. The method of claim 1, wherein the predicted source is a component of the vehicle, and the method further comprises communicating with an inspection device associated with the component, and causing the inspection device to perform a diagnostic on the component.

8. The method of claim 1, wherein the at least one operating parameter includes a type of the vehicle, and the method further comprises associating the predicted source with the type of the vehicle.

9. The method of claim 1, further comprising storing at least one of the measurement data and information related to the predicted source in a blockchain-based system.

10. A system for monitoring emissions from a vehicle, comprising:
a machine learning module including a processor configured to receive real time measurement data from an emissions sensor, the measurement data including a detected amount of a constituent of emissions from the vehicle, the machine learning module configured to input the measurement data and real time environmental monitoring data from an external monitoring device to a machine learning model, the machine learning model configured to correlate the measurement data and at least one operating parameter with a source of the detected amount of the constituent, and predict the source of the detected amount of the constituent;
an inspection device associated with a component of the vehicle, the inspection device configured to, based on the source of the detected amount of the constituent being an operating condition of the component, perform a diagnostic on the component;
one or more processors configured to communicate the predicted source and a result of the diagnostic to at least one of a vehicle user and an external entity; and
a controller configured to, based on predicting the source, autonomously control the vehicle to reduce the emissions.

11. The system of claim 10, wherein the one or more processors include a user interface configured to perform at least one of: presenting information related to the predicted source, presenting a suggestion for adjusting operation of the vehicle, and providing a route suggestion.

12. The system of claim 10, wherein the detected amount of the constituent includes at least one of: a concentration of the constituent and a chemical profile.

13. The system of claim 10, wherein the constituent of the emissions includes a pollutant, and the one or more processors are configured to compare the detected amount of the constituent to a threshold amount, and based on the detected amount exceeding the threshold amount, perform at least one of: autonomously controlling the vehicle to avoid a sensitive site, and suggesting a route to the user that avoids the sensitive site.

14. The system of claim 10, wherein the one or more processors are configured to determine a route of the vehicle based the detected amount of the constituent and the environmental monitoring data.

15. The system of claim 10, wherein the at least one operating parameter includes a type of the vehicle, and the system is configured to associate the predicted source with the type of the vehicle.

16. The system of claim 10, wherein the system is configured to store at least one of the measurement data and information related to the predicted source in a blockchain-based system.

17. A vehicle system comprising:
an emissions sensor coupled to an exhaust system of a vehicle;
a machine learning module including a processor configured to receive real time measurement data from the emissions sensor, the measurement data including a detected amount of a constituent of emissions from the vehicle, the machine learning module configured to input the measurement data and real time environmental monitoring data from an external monitoring device to a machine learning model, the machine learning model configured to correlate the measurement data and at least one operating parameter with a source of the detected amount of the constituent, and predict the source of the detected amount of the constituent;
an inspection device associated with a component of the vehicle, the inspection device configured to, based on the source of the constituent being an operating condition of the component, perform a diagnostic on the component; and
a controller configured to, based on predicting the source, autonomously control the vehicle to reduce the emissions.

18. The vehicle system of claim 17, further comprising a user interface configured to perform at least one of: presenting information related to the predicted source, presenting a suggestion for adjusting operation of the vehicle, and providing a route suggestion.

19. The vehicle system of claim 17, further comprising one or more processors configured to receive the real time environmental monitoring data from the external monitoring device.

20. The vehicle system of claim 19, wherein the one or more processors are configured to determine a route of the vehicle based the detected amount of the constituent and the environmental monitoring data.
